(19) United States
(12) Patent Application Publication
(10) Pub. No.: US 2012/0004578 A1
(43) Pub. Date: Jan. 5, 2012

(54) System and Method for Analyzing Biomechanics

(75) Inventors: Chi-Kang Wu, Taipei City (TW); Yu-Wen Chen, Tainan City (TW); Hian-Kun Tenn, Kaohsiung City (TW); Chih-Hung Huang, Xinying City (TW)

(73) Assignee: INDUSTRIAL TECHNOLOGY RESEARCH INSTITUTE, Hsinchu (TW)

(21) Appl. No.: 12/842,244

(22) Filed: Jul. 23, 2010

(30) Foreign Application Priority Data
Jul. 1, 2010 (TW) 99121749

(51) Int. Cl. A61B 5/11

(52) U.S. Cl. 600/595

(57) ABSTRACT

A system and a method for analyzing biomechanics. The biomechanics analyzing system for analyzing a motion state of an organism includes a detecting unit, a low-pass filter, a high-pass filter and a processing unit. The detecting unit disposed on a surface of a muscle of the organism detects an acceleration signal. The low-pass filter filters the acceleration signal to produce a low-frequency signal. The high-pass filter filters the acceleration signal to produce a high-frequency signal. The processing unit analyzes a motion posture or a motion frequency of the motion state of the organism according to the low-frequency signal, and analyzes a motion strength of the motion state of the organism according to the high-frequency signal.

This application claims the benefit of Taiwan application Serial No. 099121749, filed Jul. 1, 2010, the subject matter of which is incorporated herein by reference.

### BACKGROUND

1. Technical Field

The disclosure relates in general to a biomechanics analyzing system and a biomechanics analyzing method, and more particularly to a system and a method for analyzing biomechanics through the muscle motion analysis.

2. Description of the Related Art

Recently, the progress in the technology makes a lot of human works be replaced with the mechanical power so that a lot of convenience in the life are brought. However, the exercising opportunity of the human body is relatively gradually decreased. The lifestyle of the human is changed gradually from the conventional exercise life to the sedentary life, and the physical fitness of the human body will be unavoidably degraded. Among the physical fitness abilities, the evaluation and the enhancement of the cardiorespiratory function are frequently aimed, and the other physical fitness abilities are often neglected. This causes the unbalanced enhancement in the physical fitness ability, and also degrades the training effect. The muscle fitness degradation in the motion further causes the frequently seen disease of civilization. For example, the low back pain is frequently caused by the muscular problem (i.e., the muscle weakness or muscle tightness) in the motion. At present, many references have proved that the enhancement of the muscle strength is advantageous to the maintenance of the health-related physical fitness of the non-athlete and the prevention of the modern disease of civilization. Thus, an apparatus capable of monitoring the muscle state during motion is gradually needed.

In addition, the man-machine application interface is rapidly developed. Information only can be inputted to the conventional man-machine application interface through the keyboard or the mouse. However, the motion of the human body can be detected by instruments, such as an infrared detector, a gyroscope and like, which have been recently developed to input commands to the computer or playstation. However, these instruments still can only detect the simple motions, such as the up, down, left and right motions, but cannot recognize the motion of the human body so that its application is relatively restricted.

### SUMMARY

The disclosure is directed to a biomechanics analyzing system and a biomechanics analyzing method for analyzing the mechanomyogrphy (MMG) according to the acceleration signal detected by the detecting unit. Thus, the information, such as the motion posture and the motion frequency of the user, can be obtained, and different motion strengths can be distinguished.

According to a first aspect of the present disclosure, a biomechanics analyzing system for analyzing a motion state of an organism is provided. The system includes a detecting unit, a low-pass filter, a high-pass filter and a processing unit. The detecting unit disposed on a surface of a muscle of the organism detects an acceleration signal. The low-pass filter filters the acceleration signal to produce a low-frequency signal. The high-pass filter filters the acceleration signal to produce a high-frequency signal. The processing unit analyzes a motion posture or a motion frequency of the motion state of the organism according to the low-frequency signal, and analyzes a motion strength of the motion state of the organism according to the high-frequency signal.

According to a second aspect of the present disclosure, a biomechanics computerize analyzing method for analyzing a motion state of an organism is provided. The method includes the following steps. An acceleration signal is detected on a surface of a muscle of the organism. The acceleration signal is filtered to produce a low-frequency signal. The acceleration signal is filtered to produce a high-frequency signal. A motion posture or a motion frequency of the motion state of the organism is analyzed according to the low-frequency signal. A motion strength of the motion state of the organism is analyzed according to the high-frequency signal.

The above and other aspects of the disclosure will become better understood with regard to the following detailed description of the non-limiting embodiment(s). The following description is made with reference to the accompanying drawings.

### BRIEF DESCRIPTION OF THE DRAWINGS

FIG. 1 is a block diagram showing a biomechanics analyzing system according to a first embodiment of the disclosure.

FIGS. 2A to 3B are schematic illustrations showing a user wearing a detecting unit to do exercise.

FIG. 4 is a flow chart showing a biomechanics computerize analyzing method according to the first embodiment of the disclosure.

FIG. 5 shows the low-frequency signals, which are obtained in the examples of the first and second actual measurements.

FIGS. 6A and 6B respectively high-frequency signals, which are obtained in the examples of the first and second actual measurements.

FIG. 7 is a flow chart showing a biomechanics computerize analyzing method according to a second embodiment of the disclosure.

### DETAILED DESCRIPTION

### First Embodiment

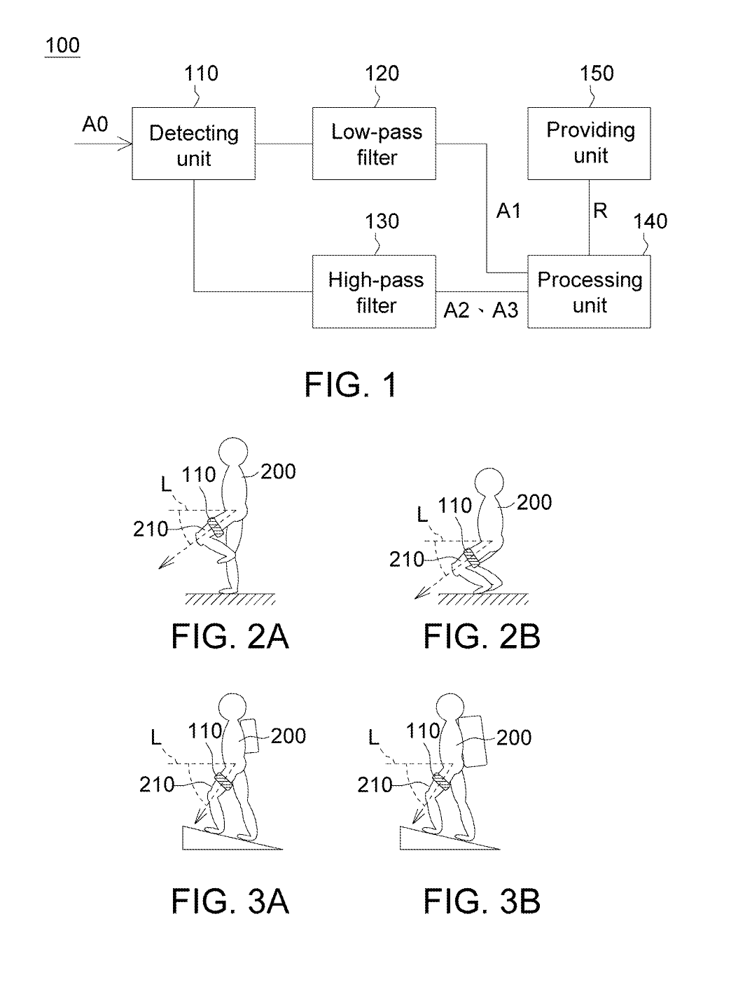

FIG. 1 is a block diagram showing a biomechanics analyzing system 100 according to a first embodiment of the disclosure. Referring to FIG. 1, the biomechanics analyzing system 100 is for detecting a motion state of an organism. The organism may be an animal, such as the human, cat, dog, horse or fish. The biomechanics analyzing system 100 includes a detecting unit 110, a low-pass filter 120, a high-pass filter 130, a processing unit 140 and a providing unit 150. The detecting unit 110 detects an acceleration signal A0 and may be, for example, a mechanical accelerometer, a piezoelectric voltage-type accelerometer, a charge-type accelerometer or a capacitive accelerometer. The low-pass filter 120 filters an electronic signal, and then let the low-frequency components pass. The high-pass filter 130 filters an electronic signal and then let the high-frequency components pass. The processing unit 140 analyzes various signals to obtain the associated information. The low-pass filter 120, the high-pass filter 130 and the processing unit 140 may be, for example, a chip, a firmware circuit or a computer readable recording medium for storing a plurality of sets of program codes. The providing unit 150, such as a hard disk, a memory card, a keyboard, a mouse or a transmission cable, provides a lot of required information.

FIGS. 2A to 3B are schematic illustrations showing a user 200 wearing the detecting unit 110 to do exercise. In FIG. 2A, the user 200 stands and lift his/her foot. The detecting unit 110 is worn on a thigh 210 of the user 200. The biomechanics analyzing system 100 (see FIG. 1) of this embodiment can analyze the angle of the thigh 210 with respect to the horizontal plane L to obtain the motion posture of the thigh 210 of the user 200. If the user 200 repeats the same motion, the biomechanics analyzing system 100 of this embodiment may also analyze its motion frequency.

In FIG. 2B, the user 200 performs the semi-crouch motion. In FIGS. 2A and 2B, the angles of the thigh 210 with respect to the horizontal plane L are similar, but the motion strength (non-explicit motion) of the muscle of the thigh 210 of FIG. 2B is greater than the motion strength of the muscle of the thigh 210 of FIG. 2A. The biomechanics analyzing system 100 (see FIG. 1) of this embodiment can further analyze the motion strength of the muscle of the thigh 210.

In FIG. 3A, the user 200 performs the hill climbing motion. The biomechanics analyzing system 100 (see FIG. 1) of this embodiment can analyze the angle of the thigh 210 with respect to the horizontal plane L to obtain the motion posture of the thigh 210 of the user 200. If the user 200 repeats the same motion, the biomechanics analyzing system 100 of this embodiment may also analyze its motion frequency.

In FIG. 3B, the user 200 also performs the hill climbing motion, but the loading of the user 200 in FIG. 3B is greater than the loading of the user 200 in FIG. 3A, so that the motion strength of the muscle of the thigh 210 in FIG. 3B is greater than that in FIG. 3A. The biomechanics analyzing system 100 (see FIG. 1) of this embodiment can further analyze the motion strength of the muscle of the thigh 210.

Of course, in addition to the thigh 210, the detecting unit 110 may also be disposed on other extremities, the head, the breast, the waist, and the position thereof does not intend to restrict the disclosure.

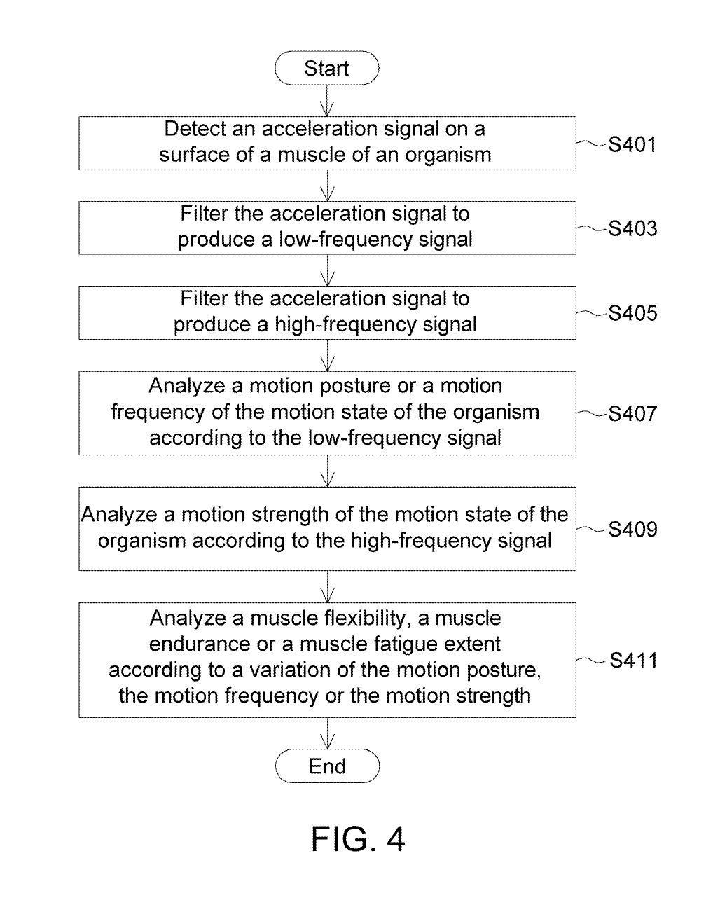

FIG. 4 is a flow chart showing a biomechanics computerize analyzing method according to the first embodiment of the disclosure. As shown in FIGS. 1 and 4, the biomechanics computerize analyzing method of this embodiment will be clearly described with reference to an actual measurement example. In one actual measurement example, the detecting unit 110 is attached to the triceps brachii of the user, and two measurements are performed. In the first measurement, the user holds a dumbbell with 7.5 kilograms and repeatedly performs the bench press raw motion. In the second measurement, the user holds the dumbbell with 15 kilograms and repeatedly performs the bench press raw motion. However, those skilled in the art may easily understand that the biomechanics analyzing system 100 of this embodiment is not particularly restricted to this flow chart, and the order and the contents of the steps may be properly adjusted.

First, in step S401, the detecting unit 110 is disposed on the surface of the muscle of the organism to detect the acceleration signal A0.

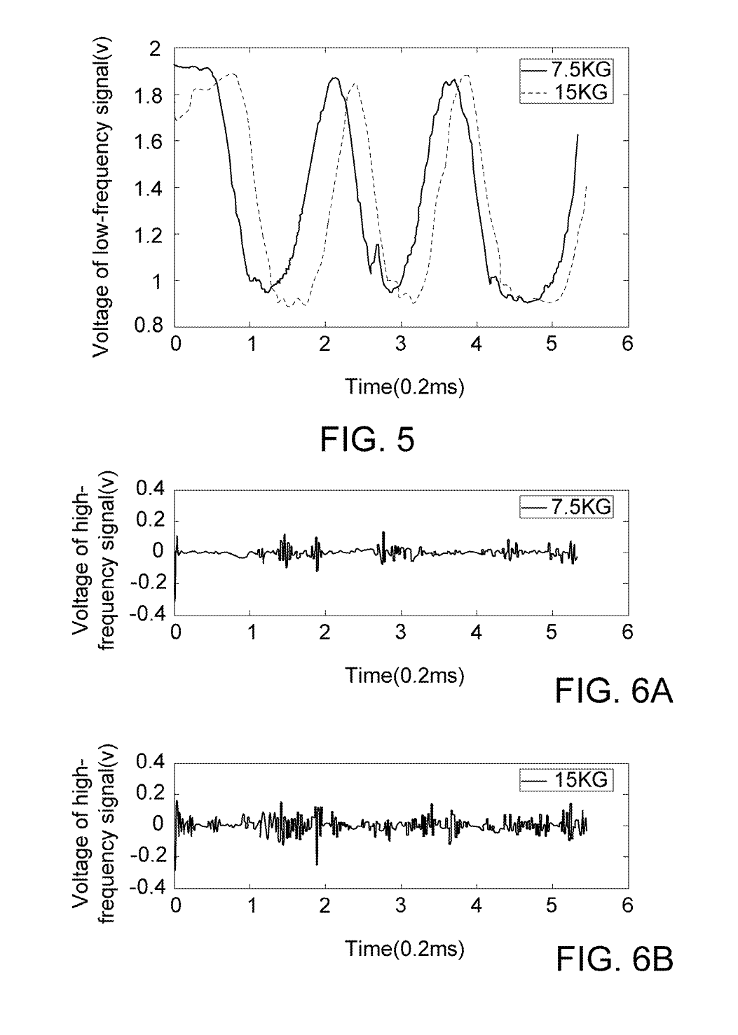

Next, in step S403, the low-pass filter 120 filters the acceleration signal A0 to produce a low-frequency signal A1. FIG. 5 shows the low-frequency signals A1, which are obtained in the examples of the first and second actual measurements.

Then, in step S405, the high-pass filter 130 filters the acceleration signal A0 to produce a high-frequency signal A2. FIGS. 6A and 6B respectively high-frequency signals A2, which are obtained in the examples of the first and second actual measurements. As shown in FIGS. 6A and 6B, it is obtained that the signal amplitude of the high-frequency signal A1 in the first measurement is smaller than that of the high-frequency signal A2 in the second measurement.

Next, in step S407, the processing unit 140 analyzes the motion posture or the motion frequency of the motion state of the organism according to the low-frequency signal A1. The processing unit 140 can analyze the angle variation during the motion process according to the signal waveform of the low-frequency signal A1, and further analyze the motion posture.

In another embodiment, the processing unit 140 may also determine the motion posture of the motion state of this organism by way of comparison according to various motion postures and the signal waveforms corresponding thereto.

In this step, the processing unit 140 further analyzes the motion frequency according to a signal frequency of the low-frequency signal A1. In general, the signal frequency and the motion frequency of the low-frequency signal A1 are the same, so the processing unit 140 only needs to calculate the motion frequency of the low-frequency signal A1 to obtain the motion frequency of the motion state of the organism.

In the example of FIG. 5, the low-frequency signals A1 in the first and second measurements have the similar signal waveforms and the similar signal frequencies. Thus, the motion postures and the motion frequencies of the user in the first and second measurements are similar to each other, respectively.

Then, in step S409, the processing unit 140 analyzes the motion strength according to a maximum value or a minimum value of a signal amplitude of the high-frequency signal A2. The processing unit 140 may determine the value of the motion strength by way of comparison according to the maximum/minimum value of the signal amplitude. In practice, the abnormal noise may also be filtered and then the signal amplitude is analyzed. In general, the motion strength is greater as the signal amplitude gets larger. For example, the signal amplitude of FIG. 6B is greater than the signal amplitude of FIG. 6A. Thus, the processing unit 140 can obtain that the motion strength of the user in the second measurement is greater than that in the first measurement.

In addition, the corresponding relationship R (see FIG. 1) between the signal amplitude and the motion strength needs not to have the directly proportional and linear relationship. In this embodiment, the corresponding relationship R between the signal amplitude and the motion strength may be stored in a storage unit in advance, and may serve as the reference for the processing unit 140 to analyze the motion strength after the provision of the providing unit 150. Consequently, the processing unit 140 may further obtain the value of the motion strength (e.g., the dumbbell with 7.5 or 15 kilograms).

Next, in step S411, the processing unit 140 further analyzes a muscle flexibility, a muscle endurance or a muscle fatigue extent according to the variation of the motion posture, the motion frequency or the motion strength. For example, the fatigue extent of the muscle can be analyzed according to the variation of the motion strength with the time under the specific motion posture and the specific motion frequency.

### Second Embodiment

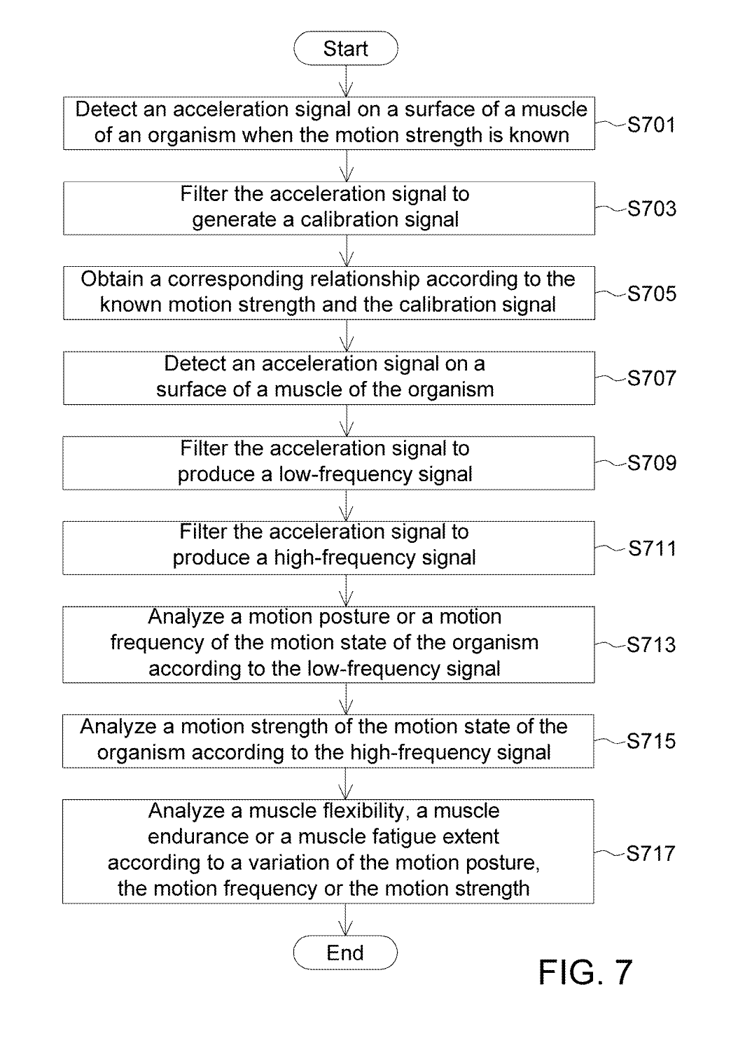

FIG. 7 is a flow chart showing a biomechanics computerize analyzing method according to a second embodiment of the disclosure. As shown in FIG. 7, the difference between the biomechanics computerize analyzing methods of the second and first embodiment resides in the method of providing the corresponding relationship R (see FIG. 1) between the signal amplitude and the motion strength, and other similar detailed descriptions will be omitted.

First, in step S701, the detecting unit 110 detects the acceleration signal A0 on the surface of the muscle of the organism when the motion strength is known. The known motion strength is, for example, that the user does not perform any motion, or the motion that the maximum strength is reached. The motion type is, for example, the isometric contraction motion or the isotonic contraction motion.

Next, in step S703, the high-pass filter 130 filters the acceleration signal A0 to produce a calibration signal A3 (shown in FIG. 1).

Then, in step S705, the processing unit 140 obtains the corresponding relationship R according to the known motion strength and the calibration signal A3. The corresponding relationship R may be stored in the storage unit.

Next, in steps S707 to S717, the actual measurement is performed on the user to obtain the measurement result. The steps S707 to S717 are similarly to the steps S401 to S411 of the first embodiment, so detailed descriptions thereof will be omitted.

According to the above-mentioned embodiments, the biomechanics analyzing system 100 and method analyze the mechanomyogrphy (MMG) according to the acceleration signal A0 detected by the detecting unit 110. Thus, the information, such as the motion posture and the motion frequency of the user, can be obtained, and different motion strengths may further be distinguished.

In addition, the computerize analyzing method adopted in the embodiment does not correspond to the electromygraphy (EMG). The detecting unit 110 of the embodiment does not need the gel to serve as the electroconductive medium so that the user cannot feel uncomfortable.

Furthermore, the biomechanics analyzing system 100 of the embodiment may be integrated in a cap-type motion kit, such as a flexible kneecap, and is thus quite convenient in the product application.

While the disclosure has been described by way of example and in terms of the exemplary embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A biomechanics analyzing system for analyzing a motion state of an organism, the system comprising:
a detecting unit, disposed on a surface of a muscle of the organism, for detecting an acceleration signal;
a low-pass filter for filtering the acceleration signal to produce a low-frequency signal;
a high-pass filter for filtering the acceleration signal to produce a high-frequency signal; and
a processing unit for analyzing a motion posture or a motion frequency of the motion state of the organism according to the low-frequency signal, and analyzing a motion strength of the motion state of the organism according to the high-frequency signal.

2. The system according to claim 1, wherein the processing unit analyzes the motion strength according to a signal amplitude of the high-frequency signal.

3. The system according to claim 2, further comprising:
a providing unit for providing a corresponding relationship between the signal amplitude and the motion strength, wherein the processing unit further analyzes the motion strength according to the corresponding relationship.

4. The system according to claim 1, wherein the processing unit analyzes the motion frequency according to a signal frequency of the low-frequency signal.

5. The system according to claim 1, wherein the processing unit analyzes the motion posture according to a signal waveform of the low-frequency signal.

6. The system according to claim 1, wherein the detecting unit is disposed on extremities of the organism.

7. The system according to claim 1, wherein the detecting unit is a mechanical accelerometer, a piezoelectric voltage-type accelerometer, a charge-type accelerometer or a capacitive accelerometer.

8. A biomechanics computerize analyzing method for analyzing a motion state of an organism, the method comprising the steps of:
detecting an acceleration signal on a surface of a muscle of the organism;
filtering the acceleration signal to produce a low-frequency signal;
filtering the acceleration signal to produce a high-frequency signal;
analyzing a motion posture or a motion frequency of the motion state of the organism according to the low-frequency signal; and
analyzing a motion strength of the motion state of the organism according to the high-frequency signal.

9. The method according to claim 8, wherein in the step of analyzing the motion strength, the motion strength is obtained according to a signal amplitude of the high-frequency signal.

10. The method according to claim 9, further comprising the step of:
providing a corresponding relationship between the signal amplitude and the motion strength,
wherein in the step of analyzing the motion strength, the motion strength is obtained according to the corresponding relationship.

11. The method according to claim 10, wherein the step of providing the corresponding relationship comprises:
detecting the signal amplitude to obtain the corresponding relationship when the motion strength is known.

12. The method according to claim 8, wherein in the step of analyzing the motion frequency, the motion frequency is obtained according to a signal frequency of the low-frequency signal.

13. The method according to claim 8, wherein in the step of analyzing the motion posture, the motion posture is obtained according to a signal waveform of the low-frequency signal.

14. The method according to claim 8, further comprising the step of:
analyzing a muscle flexibility, a muscle endurance or a muscle fatigue extent of the organism according to a variation of the motion posture, the motion frequency or the motion strength.
